(19) United States
(12) Patent Application Publication
(10) Pub. No.: US 2008/0096783 A1
(43) Pub. Date: Apr. 24, 2008

(54) Bamboo vinegar shampoo for pets and manufacturing process of the same

(75) Inventor: Feng-Huei Lin, Taipei City (TW)

Correspondence Address: ALLEMAN HALL MCCOY RUSSELL & TUTTLE LLP, 806 SW BROADWAY, SUITE 600, PORTLAND, OR, 97205-3335, US

(73) Assignee: All-Wins Biotechnology Co., Ltd.

(21) Appl. No.: 11/584,130

(22) Filed: Oct. 20, 2006

(51) Int. Cl. A61K 8/00

(52) U.S. Cl. 510/119

(57) ABSTRACT

Bamboo vinegar is a natural product for sterilization and inhibition of bacteria growth and produces no pollution during the manufacturing process, and the discarded objects after the manufacturing process produce no pollution to the environment either. The bamboo vinegar added to shampoo for a pet's body and shampoo for a pet's hair have sterilization and antibacterial effects.

### FIELD

The present invention relates to a bamboo vinegar shampoo for pets and manufacturing process of the same, more particularly to a combined solution of conventional shampoo for pets and bamboo vinegar extracted from natural bamboo having sterilization and antibacterial effects.

### BACKGROUND

With the trend of having pets for companions, the chances and frequency of human contact with animals are raising. The fur of pets, such as dogs and cats, is generally thicker then human hair and, if not cleaned properly, the pets may get infected by bacteria or may have dust mites or parasites living on their bodies. This situation sometimes leads to humans becoming infected from pets resulting in allergic reactions such as asthma or other syndromes.

The causes of allergic asthma are mostly due to contact between humans and animals. There are more and more people with allergic constitutions and the environment is full of allergens such as proteinase carried or produced by dust mites in the air and also in the bodies of humans and animals. The allergens may hide in hairs, hair roots, facial hairs, or body hairs and may cause scurf, body itchiness, or pimples on the face. The modern life style significantly increases the chances of triggering the allergy and about half of the Taiwanese families and one third of the population in Taiwan, about seven million people, have allergic syndromes. Besides seeing a doctor for proper treatment and medication, it will be more suitable to improve the living conditions for both humans and animals living with humans.

Bamboo vinegar is a naturally-extracted bio-tech product. Because many allergens can only be metabolized properly by absorbing salt, the bamboo-extracted liquid utilizes this property described above to stop or break the biological chain so that allergens can be removed naturally and gradually in a natural way. The conventional chemistry-based insecticide can enhance the insects' drug resistance and may be harmful to a human's health. One of the characteristics of bamboo-extracted liquid is based on natural mechanisms that do not enhance insects' drug resistance. Furthermore, the bamboo-extracted liquid is not harmful to both humans and animals and the effect of it lasts long.

### BRIEF SUMMARY OF THE INVENTION

The object of the present invention is to provide a shampoo for pets with sterilization and antibacterial effects. The further object of the present invention is to provide a natural shampoo for pets.

To achieve the object of the present invention, the bamboo is smoldered to a sufficient temperature so that the smoke containing the bamboo vinegar is generated. The smoke containing the bamboo vinegar generated from the smoldering process goes through a cooling pipe to condense the smoke into the liquid as so-called bamboo vinegar liquid. The bamboo, thereafter, is converted into bamboo charcoal for other applications. Over the past hundred years, the crude bamboo vinegar has been commonly mixed with adequate amounts of water to treat athlete's foot or skin illness in the country side of Taiwan and Southern China. The bamboo vinegar in the present invention is added to shampoo for a pet's body and shampoo for a pet's hair to achieve the sterilization and antibacterial effects. The bamboo vinegar is a natural antibacterial product for sterilization, which produces no pollution or discards during manufacturing. In the present invention, the bamboo vinegar is going to be added into shampoo for pet's body or shampoo for pet's hair, and then the effect of the bamboo vinegar on sterilization and inhibition of bacteria growth will be evaluated.

### BRIEF DESCRIPTION OF THE DRAWINGS

The patent or application file contains at least one figure executed in color. Copies of this patent or patent application publication with color figure(s) will be provided by the Office upon request and payment of the necessary fee.

FIG. 1 is a flow chart of an exemplary method to generate bamboo vinegar shampoo

FIG. 2 is a photograph of MARC 145 cells in a controlled group without bamboo vinegar addition.

FIG. 3 is a photograph of fluorescent MARC 145 cells in a controlled group without bamboo vinegar addition.

FIG. 4 is a photograph of the experimental group of MARC 145 cells infected by PrrsV that is a preferred embodiment of the present disclosure.

FIG. 5 is a photograph of the experimental group of fluorescent MARC 145 cells infected by PrrsV that is preferred embodiment of the present disclosure.

### DETAILED DESCRIPTION OF THE PREFERRED EMBODIMENT

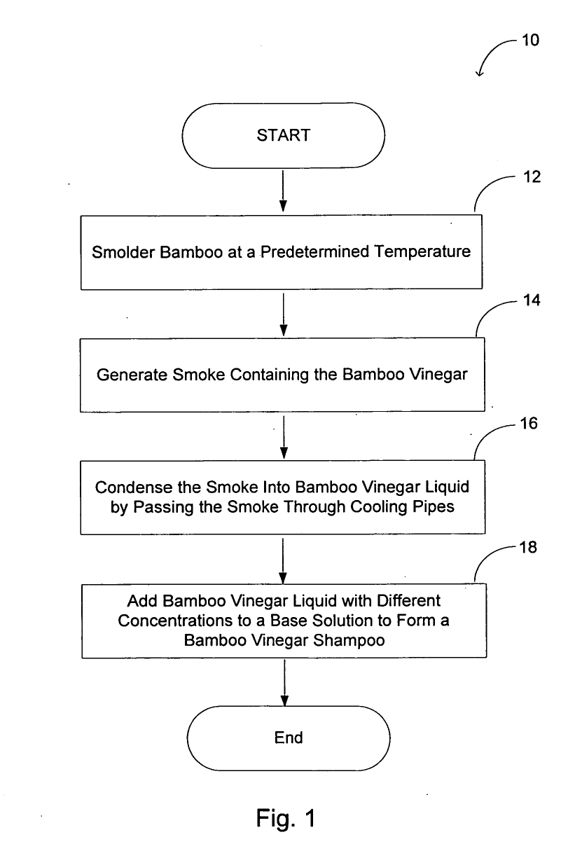

FIG. 1 shows a flow chart illustrating an exemplary method and process for creating the bamboo vinegar shampoo for pets. The method may include smoldering bamboo at a predetermined temperature at 12. In some embodiments, the temperature may be in a range from 300° C.˜400° C. Next, the method, at 14, generates smoke containing the bamboo vinegar. Then, at 16, the smoke may be condensed into bamboo vinegar by passing the smoke through a cooling pipe. Next, the method, at 18, includes adding bamboo vinegar liquid with different concentrations to a base solution to form a bamboo vinegar shampoo. The base solution of the bamboo vinegar shampoo for pets used in the preferred embodiment of the present invention includes aloe vera, Triamino Ethanol, carbopol water-soluble polymeric gel, Disodium Octaborate Terahydrate and bamboo extracts. In some embodiments, the bamboo vinegar concentrations may range from 1% to 5%.

The effect of the present invention will be evaluated by Minimal Inhibitory Concentration (MIC) and Virus-Deactivated Test.

### Minimal Inhibitory Concentration (MIC)

The minimal inhibitory concentration is the minimal concentration in which the bare eyes can observe the complete inhibition of the bacteria growth. The effect of the present invention on anti-bacteria will be evaluated by the following bacteria: Staphylococcus aureus (ATCC 29213), Pseudomons Aeruginosa (ATCC 27853), Escherichia coli (ATCC 33985), and Pityrosporum ovale (ATCC 64061), with the following steps:
1. Mueller-Hinton Broth (MHB) Culture medium, also named as Agar Culture medium, will be used in the invention. 22 grams of MHB power will be dissolved in 1 liter of de-ionized water and will be mixed homogenously.
2. Use a transfer pipette to add 2 ml of MHB to the test tube with dimension of 13×100 mm, 10 test tubes disinfected by autoclave will be prepared for the test.
3. An inoculating ring is heated for sterilization and then cooled down to room temperature, which will be used to tip out single colony from the agar plate to ensure identical genes.
4. The inoculating ring with bacteria will be put into the previous test tube with MHB solution. The bacteria concentration is 10⁸/ml for each test tube.
5. The concentration of bamboo vinegar in the present invention is 1/20, 1/40, 1/80, 1/160, 1/320, and 1/640.
6. One of the test tubes does not have bamboo vinegar therein as reference group.
7. Another test tube contains only 2 ml of the MHB solution as a blank group.
8. All test tubes are kept at the temperature of 37° C. and cultured for 24 hours.
9. After being cultured for 24 hours, 200% of cultured solution was taken out from each test tube and then placed on 96-well culture dish to analyze by an ELISA Reader with a wavelength of 600 nm to read the absorbancy.

The data shown in Table 1 is the result of this preferred embodiment in which the “Blank” refers to the base solution without bacteria, the “Control” refers to the base solution with bacteria but no bamboo vinegar, the “Origin” refers to base solution containing bacteria and bamboo vinegar liquid, and “1/20”, “1/40”, “1/80”, “1/160”, “1/320” and “1/640” refer to the base solution containing bacteria and bamboo vinegar with the dilution ratio of 1/20, 1/40, 1/80, 1/160, 1/320 and 1/640.

As shown in Table 1, the absorbancy of the bamboo vinegar dilution ratio 1/40 is the same as Blank which contains no bacteria, and this indicates the effect of sanitization. When diluting to the ratio of 1/320, the absorbancy is higher than Blank but lower than the Control which contains no bamboo vinegar. This indicates the effect of inhibition of bacteria growth. The result for Pseudomons Aeruginosa, ATCC 27853, Escherichia coli, ATCC 33985, Pityrosporum ovale, ATCC 64061 are the same and will be described as follows. After reaching the dilution ration of 1/80, the absorbancy indicates that it shows the effect of sanitization; after reaching the dilution ratio of 1/640, it still shows the effect of inhibiting bacteria from growing. In this embodiment, for Staphylococcus aureus (ATCC 29213), the MIC of the bamboo vinegar is the dilution ratio of 1/40. For Pseudomons Aeruginosa (ATCC 27853), Escherichia coli (ATCC 33985) and Pityrosporum ovale (ATCC 64061), the MIC of the bamboo vinegar is the dilution ratio of 1/640.

TABLE 1

| | Blank | Control | Origin | 1/20 | 1/40 | 1/80 | 1/160 | 1/320 | 1/640 |
|---|---|---|---|---|---|---|---|---|---|
| S. Areus | 0.55 | 0.779 | 0.072 | 0.066 | 0.067 | 0.349 | 0.425 | 0.517 | 0.685 |
| P. Areus | 0.043 | 0.678 | 0.068 | 0.070 | 0.065 | 0.079 | 0.433 | 0.476 | 0.586 |
| P. Ovale | 0.048 | 0.773 | 0.071 | 0.084 | 0.060 | 0.069 | 0.246 | 0.387 | 0.499 |
| E. Coli | 0.054 | 0.712 | 0.066 | 0.073 | 0.068 | 0.071 | 0.456 | 0.531 | 0.583 |

The Virus-Deactivated Test

The virus used in the present invention to infect MARC 145 cells is one of the viruses in porcine airway, porcine reproductive and respiratory syndrome virus (hereinafter referred to as “PrrsV”). The original concentration of the virus is 1.26×10⁶ TCID 50/ml, and is defined as 1×10⁶ TCID 50/ml.

After thawing, the virus is diluted with DMEM and centrifuged at a force of 3000 g for 5 minutes. The supernatant liquid is collected thereafter. The steps are described as follows:
1. Using a 24-well plate and inserting 10⁴/ml MARC 145 cells in each well.
2. After being cultured for 24 hours, MARC 145 cells are infected by PrrsV. The cells are washed with GKNP (Glucose Potassium Sodium Phosphate) three times. The final concentration for the test is as follows:

MARC 145: 5/mg/ml

Bamboo Vinegar: dilute to 10 and 100 times

PrrsV: 5×10⁴ TCID 50/ml
3. 0.4 ml of the processing solution is added to each well. Each of the experimental conditions are repeated 6 times with 2 wells. One well is a controlled well without virus added in and the other is an experimental well with virus added.
4. The cultured plate is placed in an incubator for one hour after being infected.
5. 0.4 ml of the medium containing 10% of the fetal bovine serum is directly added in each well; another well is rinsed with GKNP 3 times then added 0.8 ml of the medium containing 5% of the fetal bovine serum.
6. The culture plate is placed in an incubator for 5 days then rinsed with PBS 3 times.
7. Fixing the cells by using the mixing solution of acetone and methanol with volume ratio of 2:8 for 10 minutes.
8. After washed with PBS twice, 0.4 ml PBS is further added to each well and each well stays at a temperature of 4° C. to while waiting to be stained.

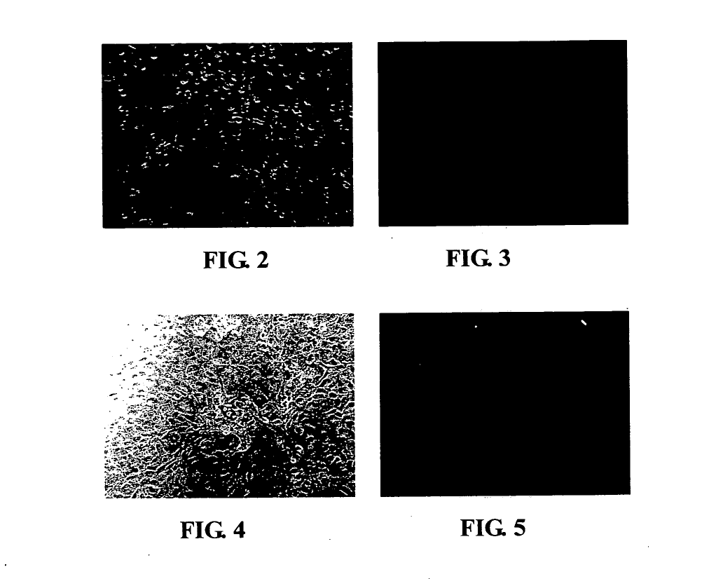

When PrrsV is immersed in the solution containing bamboo vinegar, the PrrsV will be deactivated and lose the ability to infect MARK cells that allows the MARK cells to remain the same shape and secrete the extracellular matrix. FIG. 2 is a schematic view of a preferred embodiment of the present invention, which shows that adding the vinegar solution with a dilution ratio of 1/640 can effectively deactivate pig's airway PrrsV, and the fibroblasts do not turn into round shapes, so that it will not show fluorescent green during the fluorescent staining process. FIG. 3 is a schematic view of a preferred embodiment of the present invention after processing the fluorescent staining. FIG. 4 is a schematic view of the control group of a preferred embodiment of a present invention. The group does not contain bamboo vinegar, the cells are infected by pig's airway PrrsV and shows fluorescent green. FIG. 5 is a schematic view of the control group of a preferred embodiment of the present invention after processing the fluorescent staining.

It is understood that the invention may be embodied in other forms without departing from the spirit thereof. Thus, the present example and embodiment is to be considered in all respects as illustrative and not restrictive, and the invention is not to be limited to the details given herein.

We claim:

1. A bamboo vinegar shampoo for pets, comprising a bamboo vinegar solution and a base solution, wherein the concentration of the bamboo vinegar solution is from 1% to 5%.

2. The bamboo vinegar shampoo for pets according to claim 1, wherein the bamboo vinegar solution is obtained by smoldering the bamboo to a sufficient temperature so that smoke containing the bamboo vinegar is generated then cooling down the smoke to liquid.

3. The bamboo vinegar shampoo for pets according to claim 1, wherein the base solution comprises aloe vera, Triamino Ethanol, carbopol water-soluble polymeric gel, and Disodium Octaborate Terahydrate.

4. A method to manufacture a bamboo vinegar shampoo, comprising;
smoldering bamboo at a predetermined temperature;
generating smoke containing the bamboo vinegar;
condensing the smoke into bamboo vinegar liquid by passing smoke through cooling pipes; and
adding bamboo vinegar liquid with different concentrations to a base solution to form a bamboo vinegar shampoo.

5. The method according to claim 4, wherein the predetermined temperature is in the range of 300° C.˜400° C.

6. The method according to claim 4, wherein the base solution comprises aloe vera, Triamino Ethanol, carbopol water-soluble polymeric gel, and Disodium Octaborate Terahydrate.

7. The method according to claim 4, wherein the concentration of bamboo vinegar in the bamboo vinegar shampoo is in a range of 1% to 5%.

8. A manufacturing process of bamboo vinegar shampoo comprising the steps of:
preparing a bamboo vinegar solution and a base solution; and
mixing the bamboo vinegar solution and the base solution homogeneously, wherein the concentration of the bamboo vinegar solution is from 1% to 5%.

9. The manufacturing process of bamboo vinegar shampoo according to claim 8, wherein the bamboo vinegar solution is obtained by smoldering the bamboo to 300° C.˜400° C. then cooling down the smoke to liquid.

10. The manufacturing process of bamboo vinegar shampoo according to claim 8, wherein the base solution is comprised of aloe vera, Triamino Ethanol, carbopol water-soluble polymeric gel, and Disodium Octaborate Terahydrate.
